(19) United States
(12) Patent Application Publication
(10) Pub. No.: US 2004/0126831 A1
(43) Pub. Date: Jul. 1, 2004

(54) Functionalization of immobilized proteins

(75) Inventor: William P. Van Antwerp, Valencia, CA (US)

Correspondence Address: GATES & COOPER LLP, HOWARD HUGHES CENTER, 6701 CENTER DRIVE WEST, SUITE 1050, LOS ANGELES, CA, 90045, US

(73) Assignee: MEDTRONIC MINIMED, INC.

(21) Appl. No.: 10/335,505

(22) Filed: Dec. 31, 2002

(51) Int. Cl. C12Q 1/54

(52) U.S. Cl. 435/14

(57) ABSTRACT

A method for optimizing the function of a sensor device, wherein a protein, such as an enzyme, is immobilized in a matrix, the matrix is adhered to the device and the protein comprises at least one reactive moiety. The immobilized protein is reacted with a redox agent, wherein the reacting increases the stability of the immobilized protein. The device can be sterilized after the reacting step. A typical device comprises a glucose sensor in which glucose oxidase is embedded in a polymer matrix adhered to the device, the reactive moiety comprises FADH₂, and the redox agent is the reducing agent, sodium borohydride. Also provided is a method of measuring an analyte in a tissue of a subject comprising introducing a sensor device of the invention into the tissue of the subject and detecting the signal generated by the protein. The amount of signal corresponds to the amount of analyte.

### TECHNICAL FIELD OF THE INVENTION

This invention relates generally to methods for treating a protein that has been immobilized in a device matrix to optimize the oxidative state of the protein and function of the device. The methods are suitable for use with sensors used in the detection of analytes, such as glucose.

### BACKGROUND OF THE INVENTION

Biosensors are small devices that use biological recognition properties for selective detection of various analytes or biomolecules. Typically, the sensor will produce a signal that is quantitatively related to the concentration of the analyte. To achieve a quantitative signal, a recognition molecule or combination of molecules is often immobilized at a suitable transducer, which converts the biological recognition event into a quantitative response.

The need for the continuous monitoring of biological markers (analytes) in medicine has sparked a tremendous interest in the study of biosensors in recent years. Without question, the greatest interest has been geared toward the development of sensors to detect glucose, which sensors are useful in the monitoring of diabetes. In particular, enzymatic (amperometric) glucose electrodes have been studied in more detail than other biosensors. Electroenzymatic biosensors use enzymes to convert a concentration of analyte to an electrical signal. For a review of some of the operating principles of biosensors, see Bergveld, et al., Advances in Biosensors, Supplement 1, p. 31-91, Turner ed., and Collison, et al., Anal. Chem. 62:425-437 (1990). Typically, the enzyme is immobilized onto the sensor via use of a cross-linking agent, such as glutaraldehyde.

An additional commercial application of this technology focuses on sensors that can be used to monitor fermentation reactions in the biotechnology industry. From a scientific and commercial standpoint, interest has grown beyond glucose to other analytes for the diagnosis of numerous medical conditions other than diabetes. One example of another analyte detectable via enzymatic sensors is lactate.

A typical glucose sensor works by a reaction in which glucose reacts with oxygen in the presence of glucose oxidase (GOd) to form gluconolactone and hydrogen peroxide. The gluconolactone further reacts with water to hydrolyze the lactone ring and produce gluconic acid. The H₂O₂ formed is electrochemically oxidized at an electrode as shown below (Equation 1):

H₂O₂→O₂+2e⁻+2H⁺ (I)

The current measured by the sensor/potentiostat (+0.5 to +0.7 v oxidation at Pt black electrode) is the result of the two electrons generated by the oxidation of the H₂O₂. Alternatively, one can measure the decrease in the oxygen by amperometric measurement (−0.5 to −1 V reduction at a Pt black electrode). The ultimate current detected by the sensor is proportional to the amount of glucose that reacts with the enzyme.

There is a need for an enzymatic sensor with enhanced sensing capabilities and that provides the enzyme in its most stable state. The present invention fulfills these needs and provides other related advantages.

### SUMMARY OF THE INVENTION

The invention provides a method for optimizing the function of a sensor device. The method comprises obtaining a sensor device comprising a protein immobilized in a matrix, wherein the matrix is adhered to the device and the protein comprises at least one reactive moiety. The method further comprises reacting the immobilized protein with a redox agent, wherein the reacting increases the stability of the immobilized protein. In one embodiment, the redox agent is a reducing agent. Examples of reducing agents include, but are not limited to, sodium borohydride, diborane, Fe²⁺, metals in reduced states (reduced metals), porphyrin systems, ruthenium/porphyrin complexes, and FADH₂. In another embodiment, the redox agent is an oxidizing agent. Examples of oxidizing agents include, but are not limited to, metals in oxidized states (oxidized metals), Fe³⁺, and metallo-organic porphyrins. In one embodiment, the method further comprises sterilizing the device after the reacting step.

In a typical embodiment, the protein is an enzyme. Examples of enzymes include, but are not limited to, glucose oxidase, α-hydroxy oxidase, lactate oxidase, urease, creatine amidohydrolase, creatine amidinohydrolase, sarcosine oxidase, glutamate dehydrogenase, pyruvate kinase, long chain alcohol oxidase, lactate dehydrogenase, or fructose dehydrogenase. Examples of reactive moieties include, but are not limited to, FADH₂, carbanions, and superoxide. Typically, the matrix comprises a polymer matrix. In one embodiment of the invention, the analyte is glucose, the protein is glucose oxidase, and the reactive moiety comprises FADH₂.

The invention further provides a sensor device comprising an electrode and a polymer matrix adhered to the electrode. The polymer matrix comprises a protein immobilized in the matrix, and the protein comprises a reactive moiety that has been reacted with a redox agent after the protein was immobilized in the matrix. Also provided is a method of measuring an analyte in a tissue of a subject. The method comprises introducing a sensor device of the invention into the tissue of the subject and detecting the signal generated by the protein. The amount of signal corresponds to the amount of analyte.

### DETAILED DESCRIPTION

All scientific and technical terms used in this application have meanings commonly used in the art unless otherwise specified. As used in this application, the following words or phrases have the meanings specified.

As used herein, “adhered to” or “adhered thereto” means stuck to or fused with such that a substance adhered to a surface remains substantially attached to or closely associated with the surface.

As used herein, to “increase the stability of a protein” means to place the protein in a more stable form. Typically, a protein is placed in a more stable form when its reactive moieties are put into an oxidative state that is optimal for the intended function of the protein. For example, glucose oxidase can be reacted with a reducing agent, causing the reactive moiety FAD to be reduced to FADH₂. This reduction increases the stability of glucose oxidase, and thereby optimizes the function of a sensor that is based on glucose oxidase. For a protein whose reactive moiety is a carbanion, however, reaction with an oxidizing agent would increase the stability of the protein.

As used herein, a “redox agent” refers to an oxidizing or reducing agent. An oxidizing agent is a substance that causes some other species to be oxidized or to lose electrons. A reducing agent is a substance that causes other species to be reduced or gain electrons.

As used herein, “a” or “an” means at least one, and unless clearly indicated otherwise, includes a plurality.

### Overview

Performance of an enzymatic sensor is influenced by the oxidative state of the reactive moiety in the enzyme. In the example of a glucose sensor that uses glucose oxidase, the reactive moiety is flavin adenine dinucleotide (reduced), also referred to as FADH₂. When glucose oxidase is made in air, however, the reactive moiety is primarily in the FAD or oxidized state. For ebeam analysis, it is desirable to have all of the reactive moieties in the reduced state. The preferred state will vary, of course, depending on the reactive species. For example, if a carbanion is the reactive species, then oxidation is preferred to optimize functionality of the immobilized protein.

The invention disclosed herein is based on the discovery that the functionalization of a protein immobilized in a device matrix can be enhanced by treating the protein with an oxidizing or reducing agent. The treatment with a redox agent places the reactive moieties within the protein in an oxidative state that is optimal for the device's function. This allows one to place the enzyme or other protein in its most stable form prior to sterilization of the sensor device. The enzymatic sensors of the invention provide enhanced sensing capabilities by improving the functionality of the enzyme and thereby increasing the overall sensitivity and stability of the sensors.

### Biocompatible Membranes

A glucose sensor intended for in vivo use requires that the supply of oxygen in the vicinity of the sensing element not be depleted. Additionally, the glucose should diffuse to the sensor at a controlled rate. This diffusion of the analyte to the sensor occurs through a membrane adhered to the surface of the sensor. Overall, the membrane should control the relative rates of diffusion of oxygen and glucose to the sensor so that the local concentration of oxygen is not depleted. Additionally, glucose sensors intended for in vivo use must also be biocompatible with the body. Thus, the enzyme(s) used in such sensors must be protected from degradation or denaturation, while the elements of such sensors must be protected from molecules that would foul the sensors or their accuracy will decrease over time.

In one aspect, the present invention provides a sensor device that includes a biocompatible membrane comprising an enzyme or other protein immobilized in a substrate. Typically, the enzyme or other protein is immobilized in the substrate by a cross-linking agent. The substrate is typically a polymer matrix. Suitable polymeric compositions are known in the art (see, e.g., U.S. Pat. Nos. 5,777,060 and 5,786,439, both of which are incorporated herein by reference).

### Redox Agents

Suitable redox agents for use with the invention include oxidizing and reducing agents. The selection of an oxidizing or reducing agent will depend on the reactive moieties present within the protein utilized in the sensor. The redox agent is selected to optimize function of the sensor device by placing the reactive moieties in an optimal oxidative state.

Atoms, ions, and molecules that have an unusually large affinity for electrons tend to be good oxidizing agents. Elemental fluorine, for example, is the strongest common oxidizing agent. F₂ is such a good oxidizing agent that metals, quartz, asbestos, and even water burst into flame in its presence. Other good oxidizing agents include O₂, O₃, and Cl₂, which are the elemental forms of the second and third most electronegative elements, respectively.

Compounds with unusually large oxidation states provide another good source for oxidizing agents. Examples of such oxidizing agents include the permanganate (MnO₄⁻), chromate (CrO₄²⁻), and dichromate (Cr₂O₇²⁻) ions, as well as nitric acid (HNO₃), perchloric acid (HClO₄), and sulfuric acid (H₂SO₄). These compounds are strong oxidizing agents because elements become more electronegative as the oxidation states of their atoms increase.

Good reducing agents include the active metals, such as sodium, magnesium, aluminum, and zinc, which have relatively small ionization energies and low electro-negativities. Metal hydrides, such as NaH, CaH₂, and LiAlH₄, which formally contain the H⁻ ion, are also good reducing agents. Sodium borohydride is one example of a suitable reducing agent.

Some compounds can act as either oxidizing agents or reducing agents. One example is hydrogen gas, which acts as an oxidizing agent when it combines with metals and as a reducing agent when it reacts with nonmetals. Another example is hydrogen peroxide, in which the oxygen atom is in the −1 oxidation state. Because this oxidation state lies between the extremes of the more common 0 and −2 oxidation states of oxygen, H₂O₂ can act as either an oxidizing agent or a reducing agent.

### Biosensors

Biosensors typically include a transducer, such as a protein or enzyme, that generates a signal upon contact with an analyte of interest, and is adhered to a detector, such as an electrode. For example, glucose sensors suitable for in vivo use can be prepared by depositing a membrane comprising a glucose sensitive enzyme, such as glucose oxidase, onto an electrode via an electromotive plating process. The membrane can be applied by immersion of the sensor in a bath comprising glucose oxidase, a stabilizing protein, a surfactant and a buffer for conductivity and stability of the protein solution, and the enzyme is then deposited onto the electrode potentiometrically. Alternatively, the membrane can be applied using a microelectrogravimetric plating method, such as is described in U.S. Pat. No. 6,340,021, issued Jan. 22, 2002. Methods for applying the membrane to the sensor can include a spin coating process, a dip and dry process, a microdeposition process, a jet printer deposition process, a screen printing process or a doctor blading process. Methods for preparing and applying the membrane are described in U.S. patent application Ser. No. 10/273,767, entitled “Analyte Sensors and Methods For Making Them”, filed Oct. 18, 2002.

The invention provides a sensor for measuring an analyte of interest in biological tissue, the sensor having a coating comprising a biocompatible membrane of the invention that includes an enzyme serving as a transducer that generates a signal upon contact with the analyte. The sensor can be used in vitro, or is suitable for use as an implantable biosensor or other in vivo applications. In a preferred embodiment, the analyte is glucose and the transducer is glucose oxidase. Other enzymes can serve as transducers as appropriate for the analyte of interest and examples of such enzymes include, but are not limited to, α-hydroxy oxidase, lactate oxidase, urease, creatine amidohydrolase, creatine amidinohydrolase, sarcosine oxidase, glutamate dehydrogenase, pyruvate kinase, long chain alcohol oxidase, lactate dehydrogenase, and fructose dehydrogenase.

### Methods

The invention provides a method for optimizing the function of a sensor device. The method comprises obtaining a sensor device comprising a protein immobilized in a matrix, wherein the matrix is adhered to the device and the protein comprises at least one reactive moiety. The method further comprises reacting the immobilized protein with a redox agent, wherein the reacting increases the stability of the immobilized protein.

In one embodiment, the method further comprises sterilizing the device after the reacting step. Methods of sterilization are well-known in the art, and include electron beam sterilization, other radiation methods, such as gamma radiation, and superheated steam sterilization (autoclaving). In one embodiment, electron beam sterilization is performed with a single dose of 2.0 Mrads (or 20 kGy). In other embodiments, smaller dose levels may be used if sufficient sterilization may be achieved at the lower dose, such as for example 0.5 Mrads (5 kGy). Larger doses may also be used, if the sensor device can withstand doses up to 5.0 Mrads (50 kGy). In some embodiments, the sensor device is sterilized in accordance with ANSI/AAMI/ISO 11137-1995 Sterilization of health care products—Requirements for validation and routine control—Radiation sterilization. Methods for sterilization and also the use of radiation indication stickers are described in U.S. Pat. No. 6,360,888 (and also in its divisional application Ser. No. 09/973,852, filed Oct. 9, 2001).

The invention additionally provides a method of measuring an analyte in a tissue of a subject. The method comprises introducing a sensor of the invention into the tissue of the subject, and detecting the signal generated by the enzyme (or other protein). The amount of signal generated corresponds to the amount of analyte. Preferably, the analyte is glucose and the enzyme is glucose oxidase. As described herein, other analytes and other corresponding enzymes can be used. The sensor is typically introduced into the tissue in vivo, via subcutaneous implantation, although those skilled in the art will appreciate other means for introducing the sensor into the tissue.

The above description is illustrative and not restrictive. Many variations of the invention will become apparent to those of skill in the art upon review of this disclosure. Merely by way of example a variety of solvents, membrane formation methods, and other materials may be used without departing from the scope of the invention. The scope of the invention should, therefore, be determined not with reference to the above description, but instead should be determined with reference to the appended claims along with their full scope of equivalents.

All publications, patents and patent applications mentioned in this specification are herein incorporated by reference into the specification to the same extent as if each individual publication, patent or patent application was specifically and individually indicated to be incorporated herein by reference.

What is claimed is:

1. A method for optimizing function of a sensor device comprising:
(a) obtaining a sensor device comprising a protein immobilized in a matrix, wherein the matrix is adhered to the device and the protein comprises at least one reactive moiety; and
(b) reacting the immobilized protein with a redox agent;
wherein the reacting increases the stability of the immobilized protein.

2. The method of claim 1, wherein the redox agent is a reducing agent.

3. The method of claim 1, wherein the redox agent comprises sodium borohydride, diborane, Fe²⁺, a reduced metal, a porphyrin system, a ruthenium/porphyrin complex, or FADH₂.

4. The method of claim 1, wherein the redox agent is an oxidizing agent.

5. The method of claim 1, wherein the redox agent is an oxidized metal, Fe³⁺, or a metallo-organic porphyrin.

6. The method of claim 1, wherein the protein is an enzyme.

7. The method of claim 6, wherein the enzyme is glucose oxidase, α-hydroxy oxidase, lactate oxidase, urease, creatine amidohydrolase, creatine amidinohydrolase, sarcosine oxidase, glutamate dehydrogenase, pyruvate kinase, long chain alcohol oxidase, lactate dehydrogenase, or fructose dehydrogenase.

8. The method of claim 1, wherein the reactive moiety comprises FADH₂.

9. The method of claim 1, wherein the reactive moiety comprises a carbanion or superoxide.

10. The method of claim 1, wherein the matrix comprises a polymer matrix.

11. The method of claim 1, further comprising sterilizing the device after the reacting step.

12. A sensor device comprising an electrode and a polymer matrix adhered to the electrode, wherein the polymer matrix comprises a protein immobilized in the matrix, and wherein the protein comprises a reactive moiety that has been reacted with a redox agent after the protein was immobilized in the matrix.

13. The sensor device of claim 12, wherein the protein is an enzyme.

14. The sensor device of claim 13, wherein the enzyme is glucose oxidase.

15. A method of measuring an analyte in a tissue of a subject, the method comprising introducing a sensor device of claim 12 into the tissue of the subject and detecting the signal generated by the protein, wherein the amount of signal corresponds to the amount of analyte.

16. The method of claim 15, wherein the protein is an enzyme.

17. The method of claim 15, wherein the protein is an enzyme selected from the group consisting of glucose oxidase, α-hydroxy oxidase, lactate oxidase, urease, creatine amidohydrolase, creatine amidinohydrolase, sarcosine oxidase, glutamate dehydrogenase, pyruvate kinase, long chain alcohol oxidase, lactate dehydrogenase, and fructose dehydrogenase.

18. The method of claim 15, wherein the analyte is glucose and the protein is glucose oxidase.
